(19) United States
(12) Patent Application Publication
(10) Pub. No.: US 2005/0221324 A1
(43) Pub. Date: Oct. 6, 2005

(54) Genotoxicity analysis

(76) Inventors: Michael H. Fox, Fort Collins, CO (US); Charles A. Waldren, Minami-ku Hiroshima City (JP)

Correspondence Address: SANTANGELO LAW OFFICES, P.C., 125 SOUTH HOWES, THIRD FLOOR, FORT COLLINS, CO, 80521, US

(21) Appl. No.: 10/513,489

(22) PCT Filed: May 6, 2003
(86) PCT No.: PCT/US03/14242

§ 371 Date: Jun. 3, 2005

Related U.S. Application Data
(60) Provisional application No. 60/378,422, filed on May 6, 2002.

(51) Int. Cl. C12Q 1/68; C12N 15/85

(52) U.S. Cl. 435/6; 435/455

(57) ABSTRACT

Genotoxicity testing can be carried out using a genetic hybrid cell line, for example, a CHO cell line which contains human chromosome 11. This exemplified hybrid cell line expresses human CD59 on the cell surface, and the human CD59 gene serves as a test for mutagenic agents. The hybrid cell line is grown in the presence of a test compound, and the loss of cell surface CD59 is followed using a fluorescent-labeled antibody specific for human CD59 and flow cytometry to monitor the presence or absence of labeled antibody on particular cells. Absence of the labeled antibody on the surface of the cells is indicative of a mutation in the CD59 gene such that either no CD59 protein is made or there has been a mutation which results in the loss of the antibody binding site. A test compound which causes CD59 loss is deemed to be genotoxic (i.e., mutagenic). The mutations can be point mutations, deletions, inversions, insertions, or frameshifts. The sensitivity of the assay is improved when the cells are first panned with antibody specific for the cell surface marker to remove spontaneous mutants prior to challenge with the test compound. Alternatively, or in addition, an antibiotic resistance marker is incorporated onto the same chromosome as the cell surface marker, and an antibiotic selection step precedes the challenge with the potential genotoxic composition or condition.

### CROSS REFERENCE TO RELATED APPLICATIONS

This application claims benefit of U.S. Provisional Application No. 60/378,422, filed May 6, 2002.

### ACKNOWLEDGMENT OF FEDERAL RESEARCH SUPPORT

This invention was made, at least in part, with funding from the National Institutes of Health (Grant No. NIH SBIR 1 R43 CA91566-01). Accordingly, the United States Government has certain rights in this invention.

### BACKGROUND OF THE INVENTION

The field of this invention is genotoxicity testing in mammalian cells, in particular as it relates to measuring the occurrence and/or frequency of mutant phenotypes in a hybrid cell using immunological analysis coupled with flow cytometry or other analytical techniques.

There is a need in the art for simple, rapid and economical assay methods for identifying compositions with negative aspects including, but not limited to, the ability to cause mutations or other genetic damage in mammalian cells, especially where those assay methods are fast, economical and sensitive. Such assay methods improve toxicity testing and improve the safety of materials for use in or near mammals, including humans.

### SUMMARY OF THE INVENTION

This present invention provides methods for identifying genotoxic agents by measuring loss of function in hybrid cells in which a cell surface protein is expressed via a gene carried by a chromosome heterologous to the cell (in the present specific examples). The loss of function can be determined by lack of binding of a detectable ligand specific to a cell surface protein, for example, a labeled antibody binding to the cell surface protein or a labeled second antibody specific for the antibody bound to the cell surface protein, especially a fluorescently labeled antibody or second antibody, using flow cytometry. However, other means of identifying loss of a particular function carried by the heterologous chromosome can be employed, provided that it is possible to quantitate both positive and negative cells so as to determine the frequency with which the function is lost. The hybrid cell can be derived from two different species of animal; desirably the hybrid cell is a nonhuman-human hybrid which maintains at least one human chromosome, from which at least one detectable protein, preferably a cell surface protein, is expressed. As specifically exemplified, the assay system is a flow cytometry-based mammalian cell mutation assay system using (hybrid) Chinese hamster ovary (CHO) cells which contain a human chromosome that encodes proteins not encoded by naturally occurring CHO cells, and the absence of which is indicative of loss of function of in the respective encoding gene. This assay system allows the measurement of genotoxicity in mammalian cells (as required of pharmaceutical and chemical companies by the Food and Drug Administration (FDA) and Environmental Protection Agency (EPA) in a more rapid, cheaper and more informative scheme than can be achieved with the tests now employed, reducing the time required for mutant detection from days (as is now required for all assays with mammalian cells) to hours. The human-hamster hybrid CHO A_{L} and A_{N} cells carry human chromosome 11, which encodes the CD44, CD56, CD59 and CD98 cell surface proteins. The A_{N} cells also carry the hygromycin resistance determinant on the same human chromosome (11) as the noted cell surface marker. When the hybrid cells are cultured prior to and/or during challenge with the challenge compound or condition, the background of cells which have spontaneously lost the relevant human chromosome or the function of the detectable marker, e.g. cell surface protein, is reduced.

Flow cytometry is used to measure the presence or absence of a protein(s) encoded by a human chromosome stably incorporated into a Chinese hamster cell by binding of fluorescent-labeled monoclonal antibodies to the protein(s). Mutations in the gene(s) resulting in a loss of antibody binding site (or in the loss of the entire protein) are indicated by the loss of a fluorescent signal.

In the present methods the hybrid cells are cultured in the presence of a test compound (or test condition), the test compound or condition is removed, and the cells are allowed to divide several times before the presence or absence of the target function is determined. When the frequency of loss of function cells in the population is greater in those subjected to the test compound or test condition than in cells cultured in the absence of the test compound or test condition, the test compound or condition is deemed to be genotoxic.

Also within the scope of the present invention is an improved method for genotoxicity testing wherein, prior to challenge with the test compound, the cells are treated to remove those cells which have already lost the ability to express the cell surface marker which is measured in the assay. As specifically exemplified, the cells are panned to remove those which lost the ability to express the CD59 cell surface marker. The cells are trypsinized and contacted with CD59-specific antibody, and then contacted with a solid support (e.g., a culture dish) which has been previously coated with a second antibody which specifically binds the CD59-specific antibody. This pretreatment improves the sensitivity of the assay in that the background of CD59⁻ cells is reduced by from about 10-fold to about 200-fold or more, depending on the spontaneous loss of the detectable function. Further improvement is achieved when panning and antibiotic selection are combined prior to challenge of the hybrid cells with the test composition or condition.

### DETAILED DESCRIPTION OF THE INVENTION

A genotoxic agent is one that can interact by binding covalently with genetic material. Genotoxic agents can be either direct or indirect agents. Broadly speaking, genotoxic agents include both mutagenic and clastogenic agents. In the present context, a compound, composition or condition is genotoxic when it causes mutations in at least one cell type in which it is tested or prevents expression of a target gene.

A mutagen is a chemical or physical agent that produces base-pair substitutions or small insertions or deletions of one or more base pairs in genetic material. As broadly used herein, mutations include point mutations, deletions, insertions, and chromosomal rearrangements and result in loss of function (gene expression or antigenic determinant).

A clastogen is a compound, condition or environmental agent that can cause one of two types of structural changes. A clastogen can cause breaks in chromosomes that result in the gain, loss, or rearrangements of chromosomal segments. A clastogen can also cause sister chromatid exchanges during DNA replication”. In the context of the present invention, there is a loss of function (expression of a target gene or elimination within an expressed target gene of an antigenic determinant).

Hybrid cell lines are those in which the genotype is primarily of one species, e.g., a nonhuman cell such as hamster, but there is at least one chromosome of a different species (heterologous chromosome) maintained in that cell line in a stable fashion. As specifically exemplified herein, a hamster cell line (CHO, Chinese hamster ovary) contains human chromosome 11 (CHO A_{L}). The CHO A_{N} cell line is derived from the A_{L} line by modification to contain a hygromycin resistance determinant on human chromosome 11, so that the presence of the human chromosome can be selected. Selection for the presence of the human chromosome helps eliminate false positives in the genotoxicity assay. The heterologous chromosome directs the expression of at least one protein on the cell surface, and that protein is antigenically distinct from proteins made by the cell in the absence of that heterologous chromosome.

A test compound (or composition) is one for which information concerning its ability to cause mutations is sought. The compound is tested for genotoxicity by culturing the hybrid cells and detecting the loss of the protein (or an antigenic determinant thereof) whose expression is directed by the heterologous chromosome.

A test condition is one which is analyzed for the ability to cause mutation in a hybrid cell line as described herein. A test condition can be, but not limited to, irradiation with ultraviolet light, visible light, radio waves, x rays, gamma rays, shortwave, electromagnetic or microwave irradiation, for example, or it can be high or low temperature, among others.

The methods of the present invention are important in that they allow the testing for genotoxicity of chemical and physical agents using a mammalian cell system. Manufacturers of drugs and chemicals are required to test them for genotoxicity using a variety of assays, including mammalian cells. These methods can be used for the genotoxicity testing, and the improved economics and shorter times can save the drug and chemical companies significant amounts of money.

The assay methods of the present invention are more sensitive than alternative mammalian cell assays. They are also more rapid and much less expensive to use than alternative assays. It allows reduction in the time and cost of testing drugs and other agents, which could be very important for large drug and chemical manufacturers.

The A_{L} hybrid was derived almost 30 years ago by fusing a CHO cell with a normal human fibroblast (T. T. Puck, P. Wuthier, C. Jones, and F. T. Kao. 1971. Lethal antigens as genetic markers for study of human linkage groups. Proc. Natl. Acad. Sci. USA 68, 3102-3106). Subclones were selected that stably retained a single human chromosome 11. This human chromosome 11 (and all other human chromosomes 11) was subsequently shown to encode a series of surface antigens whose map positions on the chromosome are known (C. A. Waldren. 1983. Mutational analysis in cultured human-hamster hybrid cells, In: Chemical Mutagens: Principles and Methods for Their Detection, Vol. 8, (F. J. de Serres., Ed.), pp. 235-260, Plenum Publ Corp., New York; C. Waldren, L. Correll, M. A. Sognier, and T. T. Puck. 1986. Measurement of low levels of x-ray mutagenesis in relation to human disease. Proc. Natl. Acad. Sci. USA 83, 4839-4843; C. A. Waldren and C. Jones, 1981. Chromosome loss and damage as measured by biological markers, In: Health Risk Analysis: Proceedings of the Third Life Sciences Symposium, (C. R. Richmond, P. J. Walsh, and E. D. Copenhaver., Eds.), pp. 333-343, The Franklin Press, Philadelphia, Pa.). Since only one or a few genes located at the very tip of the short arm of chromosome 11 are required for survival of the hybrid, mutations ranging from a point mutation to loss or rearrangement of chromosomal fragments of greater than 160 Mbp on chromosome 11 can be detected (S. M. Kraemer, D. B. Vannais, A. Kronenberg, A. Ueno, and C. A. Waldren. 2001. Gamma-Ray Mutagenesis Studies in a New Human-Hamster Hybrid, A(L)CD59(+/−), which has Two Human Chromosomes 11 but is Hemizygous for the CD59 Gene. Radiat. Res. 156, 10-19). The standard assay for mutation using these cells is based on complement-mediated cytotoxicity. Cells are labeled with antibodies against specific surface antigens (usually CD59) and then treated with rabbit complement. Cells which express the surface antigen(s) are killed by the complement, but cells which are mutated and do not express the surface antigen(s) survive. This has allowed for sensitive quantification of the mutagenic activity of standard “point” mutagens, e.g. ethyl-methane sulfonate (EMS), N-methyl N-nitrosoguanidine (MN NG) and UV, as well as clastogens like mitomycin C and ionizing radiations (C. A. Waldren, C. A. 1983, supra; C. Waldren, C. 1986; C. Waldren, C. Jones, and T. T. Puck. 19790. Measurement of mutagenesis in mammalian cells. Proc. Natl. Acad. Sci. USA 76, 1358-1362; N. Matsukura, J. Willey, M. Miyashita, B. Taffe, D. Hoffmann, C. Waldren, T. T. Puck, and C. C. Harris. 1991. Detection of direct mutagenicity of cigarette smoke condensate in mammalian cells. Carcinogenesis 12, 685-689; S. M. McGuinness, M. L. Shibuya, A. M. Ueno, D. B. Vannais, and C. A. Waldren. 1995. Mutant quantity and quality in mammalian cells (AL) exposed to cesium-137 gamma radiation: effect of caffeine. Radiat. Res. 142, 247-255; T. K. Hei L.-J. Wu, S.-X. Liu, D. Vannais, C. Waldren, and G. Randers-Pehrson. 1997. Mutagenic effects of a single and an exact number of alpha particles in mammalian cells. Proc. Natl. Acad. Sci. USA 94, 3765-3769).

We have also shown that such non-genotoxic carcinogens as asbestos and arsenic are, in fact detectable as quite strong mutagens, producing mainly chromosomal mutations in A_{L} cells (T. K. Hei, S. X. Liu, and C. Waldren, Mutagenicity of arsenic in mammalian cells: role of reactive oxygen species. 1998. Proc. Natl. Acad. Sci. USA 95, 8103-8107; T. K. Hei, C. Q. Piao, Z. Y. He, D. Vannais, and C. A. Waldren. 1992. Chrysotile fiber is a strong mutagen in mammalian cells. Cancer Res. 52, 6305-6309).

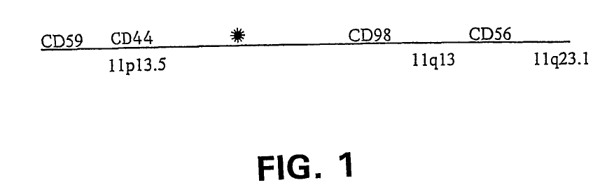

The CD59 gene maps on the short arm at 11p13.5; other genes of interest such those encoding CD98 (also known as SLC3A2) is located on the long arm at 11q13 (FIG. 1). The CD59 gene has been cloned (D. Vannais, M. White, M. McGraw, A. Davies, A. Wilson, T. Hei, and C. Waldren. 1998. Intragenic mutations in gene MCI=CD59 can now be analyzed in human-hamster hybrid AL cells. Radiat. Res. 106). Its exact size is known and PCR primers have been made which can be used to define intragenic mutations (S. M. Kraemer et al. 2001. supra). The CD56 (also known as NCAM1) gene is located at position 11q23.1 on chromosome 11. The gene located at 11p15.5 is an essential gene in the hybrid cell line, making the loss of human chromosome a lethal event. Furthermore, a new cell line (CHO A_{N}) has been generated; this cell line has the hygromycin gene stable incorporated in the long arm of chromosome 11. Spontaneous loss of chromosome 11 can be selected against by adding neomycin to the cultures to reduce the level of background mutants (C. A Waldren, B. Failed, M. Braden, R. D. Parker, and D. Vannais. 1992. The use of human repetitive DNA to target selectable markers into only the human chromosome of a human/hamster hybrid cell line (AL). Somat. Cell Mol. Genet 18, 417-422).

We have developed a flow cytometric assay based on CHO A_{L} cells (or CHO A_{N} cells) that allows simultaneous detection of both point and chromosomal mutations and is much more rapid and less expensive than the traditional A_{L} assay based on complement-mediated cytotoxicity. The assay is similar to the A_{L} assay discussed above except that the presence or absence of the surface antigens such as CD44, CD59, CD56 or CD98 is measured by binding of specific monoclonal antibodies. Monoclonal antibodies specific for these markers are commercially available from Ancell Corporation, Bayport, Minn.; Research Diagnostics Inc., Flanders, N.J.; Biomedia, Foster City, Calif.; Sigma Chemical Co., St. Louis, Mo., among others. We have demonstrated that the flow cytometry based method is highly sensitive and linear, and it can readily detect mutations induced by ionizing radiation and chemical agents such as MNNG. There are several advantages of this method over the cytotoxicity-based A_{L} assay and the mouse lymphoma assay (MLA). First, the flow cytometry assay does not depend on colony growth, so the 7-10 day period required for colonies to form in the traditional assays is eliminated. Only a few hours are required to carry out the antibody labeling procedures and the flow cytometry analysis. Second, it is much less expensive because it does not require labor-intensive cell culture for colony growth and counting of colonies. Also it does not require expensive and unreliable rabbit serum complement. Third, the fully developed assay will be able to distinguish between small and large mutations by measuring the presence or absence of two or more antigens simultaneously. Fourth, the A_{L} cells are extremely robust and easy to handle with a generation time of about 12 hr, so the expression period required before analysis of mutations is relatively short, desirably 7 to 12 days, advantageously 9 days. This method is also improved over a prior art method which relies on magnetic separation of expressing and non-expressing cells.

Testing of drugs by pharmaceutical and chemical companies is a multi-billion dollar enterprise which includes both in-house testing and testing by independent companies. We have identified at least 10 commercial companies that use the MLA test (an alternative mammalian cell mutation assay system). Typical costs cited by one company range from $2000-4000 for a preliminary screening to $25,000 for a full fledged GLP (Good Laboratory Practices) assay suitable for submission to the United States FDA. The turn-around time for a full-fledged assay is about 8 weeks. Further development and validation of the flow cytometry based assay could cut the time in half for these assays and reduce the cost by at least 50%. Thus, the commercial potential for a more rapid, less expensive and more sensitive assay is enormous.

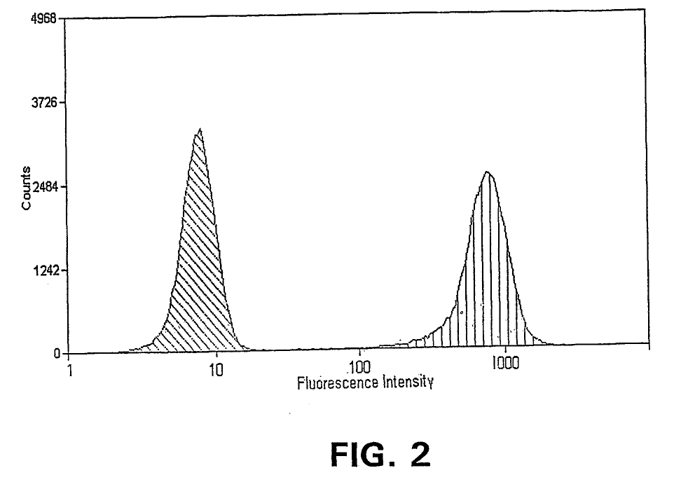

An example of the separation between positive cells containing the CD59 antigen (A_{L}) and negative cells (parental strain which does not contain human chromosome 11) is shown in FIG. 2. The peaks are separated by over two orders of magnitude.

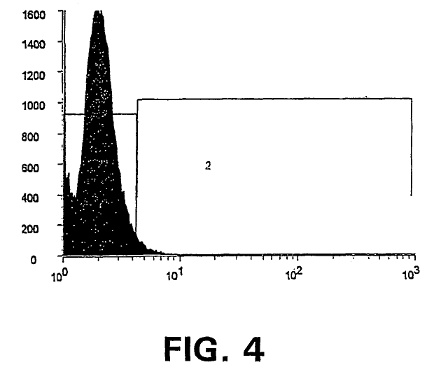

By using careful handling procedures and gating the cells on light scatter and time of flight, we have achieved measurements of 0.03% of negatives within the positive population (FIG. 4) when regions were set to include >97% of negative cells (FIG. 4). This is close to the background level of mutants usually observed within the population of positive cells.

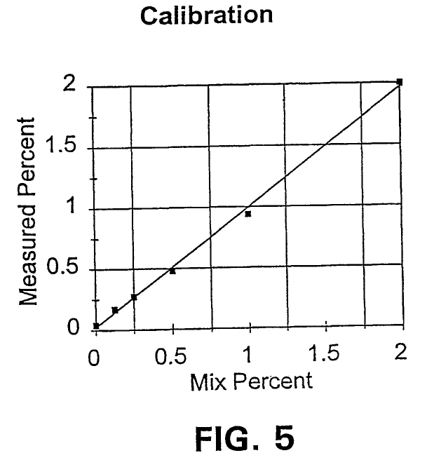

To test the accuracy of measuring a small mutant population, we did a number of mixing experiments in which various fractions of negative cells were mixed in with positive cells. Starting with an initial mixture of 2% negatives, serial two-fold dilutions were made to a minimum of 0.125% negatives. These mixed samples were then stained with antibodies against CD59 and analyzed. As shown in FIG. 5, flow cytometry measurements of the percent of mixed cells is highly linear when compared to the actual mixed percentage. The slope is 0.97 with an R-squared value of 0.998 and an intercept of 0.02 percent. These results demonstrate that flow cytometry has the intrinsic sensitivity and linearity to be useful for measuring mutant fractions induced by various treatments.

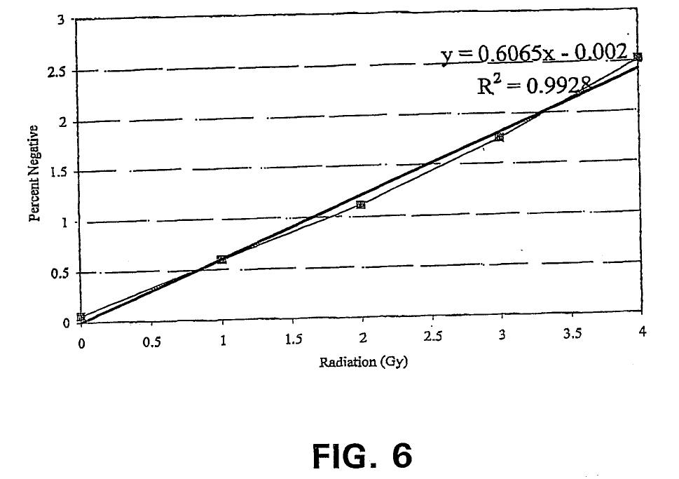

These calibration results are convincing that flow cytometry has the sensitivity needed to measure mutant yields induced in cells by different agents. In order to actually measure mutants instead of just mixed cell populations, we irradiated populations of A_{L} cells with doses from 0 to 4 Gy, then grew the cells for 9 days with subculturing as needed to allow the induced mutants to regain normal growth and to lose the expression of CD59 on the surface of the cells. Three independent samples were irradiated and subcultured for each dose. The samples were then processed for antibody staining and flow cytometry analysis, using commercially available antibody and well known techniques. Two independent experiments have been done with very similar results. The results shown in FIG. 6 represent one experiment with 3 independent samples for each dose point. There is a linear dose response for mutant yield as measured by flow cytometry. The mutant yield at 0 dose is somewhat high compared to historical controls in the standard complement-mediated assay and to other results for unirradiated control cells using flow cytometry. Without wishing to be bound by any particular theory, this is believed due, at least in part, to the fact that these cells were in culture for several months and the background level of mutants was increasing. These experiments are being repeated using cells which have been panned to reduce the background mutation frequency to determine whether a lower yield at 0 dose can be obtained. Results shown in FIG. 5 indicate that we can measure a background level of mutations of only about 0.02%. The slope of the curve is quite similar to published results using the complement-mediated cytotoxicity assay.

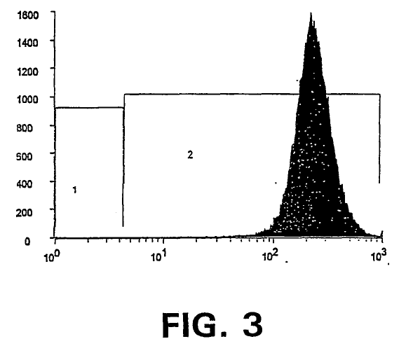

Because the separation of the positive and negative cells is so great (see FIGS. 3 and 4), the assay can be fine tuned for greater sensitivity. We have successfully measured 0.03% negative cells in a background of positive cells (for CD59) when the regions were set to include >97% of the negative cells.

Flow cytometry measurements are highly linear for mixtures of positive and negative cells when compared to the calculated percentages of positive and negative cells. The slope is 0.97 with an R-squared vale of 0.998 and an intercept of 0.2% (FIG. 5).

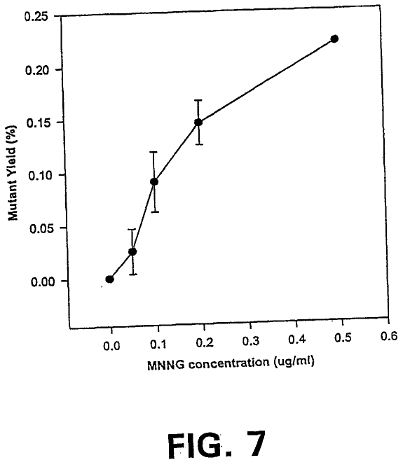

There is a linear dose-response curve for mutant yield as measured by flow cytometry after radiation of the hybrid cells (FIG. 6). The measured background mutant level in this experiment was about 0.1%. The assay for chemically induced mutations is very sensitive, measuring a significant increase in mutant yield at concentrations of 0.1 μg/ml MNNG (FIG. 7). These results demonstrate that the flow cytometry mutation assay is robust in the sense that it is sensitive to mutations induced by both radiation and alkylating agents. Thus, it has been demonstrated that the methods of the present invention are applicable to clastogenic and other mutagenic agents.

We have also measured mutant yield at the CD59 locus induced by MNNG (FIG. 7). CHO AL cells were treated with various concentrations of MNNG for 16 hr at 37° C. The mutagenized populations were then grown up for 10 days, stained with antibody against CD59 and analyzed by flow cytometry as described. The results shown are based on 2 separate analyses of the same experiment. These results clearly show an increase in mutations with increasing concentration of MNNG. They also indicate that the assay is very sensitive, measuring a significant increase in mutant yield at concentrations of 0.1 μg/ml of MNNG. These results also show that the flow cytometry mutation assay is robust in the sense that it is sensitive to mutations induced by both radiation and alkylating agents.

The results described herein above have been improved by panning the hybrid cells prior to use in genotoxicity testing studies by panning to remove those cells which, prior to challenge with a potential genotoxic agent, have lost the ability to express CD59. The cells are trypsinized, contacted with a commercially available CD59-specific mouse antibody and the bound via the CD59-specific mouse antibody to a surface coated with antibody specific for mouse antibody. This pretreatment panning lowers the background of CD59⁻ cells and increases the sensitivity of the assay from about 10-fold to 200-fold or greater, depending on the spontaneous loss of CD59 expression.

Culture of the hybrid cells prior to challenge with the test compound or test condition in the presence of an antibiotic, where a resistance determination is carried on the same heterologous chromosome as the expression product to be detected, serves to improve the sensitivity of the genotoxicity testing methods by reducing the background of cells which have spontaneously lost the heterologous chromosome. This antibiotic selection step can be used with or without the panning step. Where these two are combined, the results are improved significantly (at least 2-fold) over use of either selection alone.

Monoclonal or polyclonal antibodies, preferably monoclonal, specifically reacting with a protein of interest can be made by methods well known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Willey Interscience/Greene Publishing, New York, N.Y. Also, recombinant immunoglobulins may be produced by methods known in the art, including but not limited to the methods described in U.S. Pat. No. 4,816,567. Monoclonal antibodies with affinities of 10⁸ M⁻¹, preferably 10⁹ to 10¹⁰ or more are preferred.

Antibodies specific for particular proteins are useful, for example, as probes for screening for loss of a functional gene encoding the particular protein, in the context of the present invention. Desirably, the antibodies are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. United States patents describing the use of such labels include but are not limited to U.S. Pat. Nos. 3,817,837; 3,850,752; 3,939,350; 3,996,345; 4,277,437; 4,275,149; and 4,366,241. Fluorescent agents are especially useful in applications using fluorescence activated cell sorting.

The descriptions provided herein are for illustrative purposes, and are not intended to limit the scope of the invention as claimed. Any variations in the exemplified methods which occur to the skilled artisan are intended to fall within the scope of the present invention.

All references cited in the present application are incorporated in their entirety herein by reference to the extent not inconsistent herewith.

### Materials and Methods

Cell Culture

CHO A_{L} cells were cultured in F12 medium at 37° C. at 5% CO₂ in an incubator. Parental (CHO) cells without human chromosome 11 were used as a negative control. Cells were passaged every three days to avoid confluence. To reduce background mutations, cells were either panned by using a CD59-specific antibody or stained and sorted for CD59 positive cells as described below. When A_{N} cells are used, they are desirably cultured in medium containing 800 μg/ml neomycin before challenge with the test compound or test condition to select against cells which have spontaneously lost human chromosome 11. The antibiotic selection can be used instead of panning or in addition to panning.

Antibody Staining

Cells were stained using 50 μl and phycoerythrin-labeled monoclonal antibody specific for human CD59 per 1×10⁶ cells.

Flow Cytometry

Cells were analyzed using a Coulter EPICS V cell sorter at 488 nm with a 515 SP and 575 LP filters. Cellular debris was removed by gating on Forward Scatter vs. Side Scatter. A total of 50,000 cells were collected per sample. Cells which expressed CD59 were brightly stained, whereas mutants lacking reactivity with the CD59-specific antibody were dim. Gates were set so that 97% of the negative control parental (CHO) cells were counted.

Calibration

To ensure test sensitivity, CHO A_{L} cells were mixed with parental CHO cells at concentrations ranging from 0.0125% to 0.5%. The mixed samples were then stained with the labeled CD59-specific antibody and analyzed using flow cytometry. FIG. 5 shows typical results for assay calibration.

Chemical Induced Mutations

CHO A_{L} cells were plated and 3 hr later MNNG was added. After 3 hr incubation, the medium was aspirated, the cells were washed with sterile phosphate buffered saline, and fresh F12 medium was added to the flasks. Cells were then grown for 7-12 days, (desirably about 9 days) and analyzed by antibody binding and flow cytometry.

Radiation Induced Mutations

CHO A_{L} cells were plated in T75 flasks and then irradiated to reach a maximum dose of 80% toxicity. After a survival curve was developed, the cells were then irradiated at doses from 0 to 4 Gy, cultured for 7 to 12 days (desirably about 9 days), contacted with the labeled CD59-specific antibody and analyzed by flow cytometry as described above.

Panning

Panning is used to significantly reduce background marker loss in the A_{L} or A_{N} cells or other hybrid cells. For A_{L} or A_{N} cells, non-tissue (non-coated) culture plates, 100 mm, i.e. those not pre-coated, are incubated with goat anti-mouse IgM antibody in PBS 10 ml, 20 μg/ml, for 2 hours at room temperature to allow the IgM to bind to the plastic. The plates are then washed 3 times with PBS to remove excess, nonadhered IgM, and then they are filled with 1% fetal bovine serum (FBS) in phosphate buffered saline (PBS) (FBS/PBS) (5 ml) and stored at 4° C. until use.

Approximately 4×10⁶ A_{L} or A_{N} cells are trypsinized, harvested in 2% FBS/PBS, collected by centrifugation and resuspended in 0.2% Anti-CD59/2% FBS/PBS at a cell concentration of about 2×10⁶ per ml to allow binding of the Anti-CD59 antibody to cells which express the marker. After 30 min incubation with antibody at 4° C., the cells are rinsed with 2% FBS/PBS 3 times and then diluted in 2% FBS in PBS to give 2.5×10⁶ in 5 ml in a single plate. FBS/PBS is removed from the treated plates and then 5 ml cell suspension is added to the plates. The cells are incubated for 2 hours at 4° C., then the PBS is aspirated, removing floating cells. The plates are gently rinsed 3 times with 5 ml cold 2% FBS/PBS to remove unattached CD59⁻ cells (negatives). Complete medium is then added to the plates and pipetted forcefully several times to remove the CD59⁺ cells from the plates. The cells are then collected, passaged twice and frozen for use. At this point, the cells are very low in background CD59⁻ cells and are ready for use in genotoxicity testing studies. Where a different cell surface marker is to be monitored, the appropriate antibody is used in the selection prior to contacting with the composition or condition being tested for genotoxicity, and then the same antibody is used to measure genotoxicity (mutagenicity and/or clastogenic activity).

1. A method for genotoxicity testing, said method comprising the steps of (a) culturing a hybrid cell line derived from a first species and a second species, wherein said hybrid cell line stably maintains at least one chromosome of the first species and wherein said cell line expresses at least one cell surface protein derived from the first species, in the presence of a test compound or a test condition for from about four to about twenty cell divisions; (b) optionally subsequently culturing said cells in the absence of the test compound or test condition; (c) contacting the cells cultured in step (a) or step (b) with a detectable ligand specific for the first species cell surface protein; (d) subjecting the cells contacted with detectable ligand in step (c) to a sorting process to quantitate cells to which the labeled antibody has not bound and quantitating the cells to which the labeled antibody has bound; and (e) identifying the test compound or test condition as a genotoxic agent when there is a greater number of cells to which antibody has not bound after culture with the test compound or condition than there is when the cells have been cultured in the absence of a test compound or genotoxic compound without exposure to said composition or said condition.

2. The method of claim 1, wherein the detectable ligand comprises at least one antibody specific for said cell surface protein.

3. The method of claims 1 or 2, wherein the hybrid cell line is a human-non-human animal hybrid cell line.

4. The method of claim 3, wherein the hybrid cell line is a human-hamster hybrid cell line.

5. The method of claim 4, wherein the human-hamster hybrid cell line is a CHO AL cell line.

6. The method of any of claim 1, wherein said hybrid cell line expresses at least one antibiotic resistance gene, wherein said antibiotic resistance gene and said first species cell surface protein gene resides on the at least one first species chromosome, and wherein prior to step (a), said cell line is cultured in the presence of the antibiotic to which the at least one antibiotic resistance gene confers resistance.

7. The method of claim 4, wherein the human hamster hybrid cell line is a CHO AN cell line.

8. The method of any of claim 1, wherein the labeled antibody is a fluorescent-labeled antibody.

9. The method of any of claim 3, wherein the cell surface protein is a human protein selected from the group consisting of CD44, CD56, a CD59 and CD98.

10. The method of claim 8, wherein the cells are sorted by fluorescence activated cell sorting.

11. The method of any of claim 1, wherein prior to the step (a), said method comprises the step of removing cells which do not express the cell surface protein.

12. The method of claim 10, wherein the step of removing comprises contacting the hybrid cells with a first antibody specific for the cell surface protein and then contacting with a second antibody specific for said first antibody, wherein said second antibody is bound to a solid support.

13. The method of claim 10, wherein prior to step (a), the hybrid cells are cultured in the presence of an antibiotic to which resistance is conferred by an antibiotic resistance gene linked to the cell surface protein gene in an amount and for a time sufficient to kill cells which have lost ability to express the antibiotic resistance gene.
